(19) United States
(12) Patent Application Publication
(10) Pub. No.: US 2017/0173034 A1
(43) Pub. Date: Jun. 22, 2017

(54) COMBINATION OF A JAK INHIBITOR AND A SYK INHIBITOR FOR TREATING CANCERS AND INFLAMMATORY DISORDERS

(71) Applicant: Gilead Sciences, Inc., Foster City, CA (US)

(72) Inventor: Julie A. Di Paolo, San Francisco, CA (US)

(21) Appl. No.: 15/380,937

(22) Filed: Dec. 15, 2016

Related U.S. Application Data
(60) Provisional application No. 62/421,714, filed on Nov. 14, 2016.
(60) Provisional application No. 62/269,076, filed on Dec. 17, 2015.

(51) Int. Cl. A61K 31/541; A61K 31/4985; A61K 31/5377

(52) U.S. Cl. CPC A61K 31/541; A61K 31/5377; A61K 31/4985

(57) ABSTRACT

Provided herein are methods and pharmaceutical compositions for the treatment of inflammatory disorders comprising filgotinib and a Syk inhibitor.

### CROSS REFERENCE TO RELATED APPLICATIONS

This application claims the benefit under 35 U.S.C. §119(e) of U.S. Provisional Application No. 62/421,714, filed Nov. 14, 2016, and U.S. Provisional Application No. 62/269,076, filed Dec. 17, 2015, the entireties of which are incorporated herein by reference.

### BACKGROUND

Janus kinase (JAK) is a family of intracellular, nonreceptor tyrosine kinases that transduce cytokine-mediated signals via the JAK-STAT pathway. Filgotinib is a JAK1 selective inhibitor.

Spleen tyrosine kinase (SYK) is a member of the Syk family of tyrosine kinases, which are non-receptor cytoplasmic tyrosine kinases that share a characteristic dual SH2 domain separated by a linker domain.

### SUMMARY

Described herein are methods of treating inflammatory disorders and cancers comprising administering filgotinib, or a pharmaceutically acceptable salt, solvate, polymorph, or metabolite thereof, and a Syk inhibitor, to a patient in need thereof.

In some embodiments, provided herein is a method of treating an inflammatory disorder in a human in need thereof, comprising administering to the human:
(i) a therapeutically effective amount of filgotinib or a pharmaceutically acceptable salt, solvate, polymorph, or metabolite thereof; and
(ii) a therapeutically effective amount of a Syk inhibitor.

The present disclosure, in another embodiment, provides a composition for use in the treatment of an inflammatory disorder, the composition comprising: (i) filgotinib or a pharmaceutically acceptable salt, solvate, polymorph, or metabolite thereof; and (ii) a Syk inhibitor.

In some embodiments, the inflammatory disorder is systemic lupus erythematosus, graft versus host disease, myestenia gravis, rheumatoid arthritis, acute disseminated encephalomyelitis, idiopathic thrombocytopenic purpura, multiple sclerosis, Sjogren's syndrome, psoriasis, autoimmune hemolytic anemia, asthma, ulcerative colitis, Crohn's disease, irritable bowel disease, or chronic obstructive pulmonary disease. In some embodiments, the inflammatory disorder is rheumatoid arthritis. In some embodiments, the inflammatory disorder is systemic lupus erythematosus. In some embodiments, the inflammatory disorder is graft versus host disease.

In some embodiments, the present disclosure provides a co-formulation comprising: (i) filgotinib or a pharmaceutically acceptable salt, solvate, polymorph, or metabolite thereof; (ii) a Syk inhibitor; and (iii) a pharmaceutically acceptable carrier.

In some embodiments, the Syk inhibitor is entospletinib or a pharmaceutically acceptable salt, solvate, or polymorph thereof.

In some embodiments, the Syk inhibitor is a compound of Formula (I):

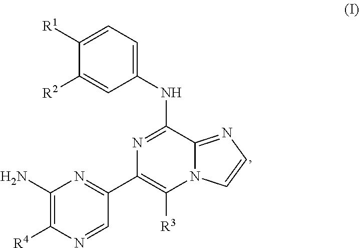

or a pharmaceutically acceptable salt, pharmaceutically acceptable co-crystal, pharmaceutically acceptable ester, stereoisomer, or tautomer thereof,
wherein:

### R¹ is:

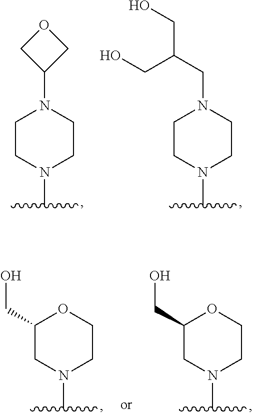 [SMILES: *N1CCN(C2COC2)CC1 ; *N1CCN(CC(CO)CO)CC1 ; *N1CCO[C@@H](CO)C1 ; *N1CCO[C@H](CO)C1]

wherein [symbol] indicates the point of attachment to the remainder of the compound of Formula (I),
R² is H or 2-hydroxyethoxy,
R³ is H or methyl, and
R⁴ is H or methyl.

### DESCRIPTION OF THE DRAWINGS

FIG. 1: Rat ankle diameter over time in the rat type II collagen-induced arthritis model. The graph shows mean±SEM.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

Provided herein are methods or treating inflammatory disorders and cancers in a human in need thereof, comprising administering to the human a therapeutically effective amount of filgotinib or a pharmaceutically acceptable salt, solvate, polymorph, or metabolite thereof, and a therapeutically effective amount of a Syk inhibitor. Provided herein are also compositions (including pharmaceutical compositions, formulations, co-formulations, or unit dosages), articles of manufacture and kits comprising filgotinib or a pharmaceutically acceptable salt, solvate, polymorph, or metabolite thereof, and a Syk inhibitor.

### Definitions

As used in the present specification, the following words, phrases and symbols are generally intended to have the meanings as set forth below, except to the extent that the context in which they are used indicates otherwise.

Reference to the word “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, reference to “the compound” includes a plurality of such compounds, and reference to “the assay” includes reference to one or more assays and equivalents thereof known to those skilled in the art.

Reference to “about” a value or parameter herein includes (and describes) embodiments that are directed to that value or parameter per se. The term “about X” thus includes description of “X”. In certain embodiments, the term “about” includes the indicated amount±10%. In other embodiments, the term “about” includes the indicated amount±5%. In certain other embodiments, the term “about” includes the indicated amount±1%.

Recitation of numeric ranges of values throughout the specification is intended to serve as a shorthand notation of referring individually to each separate value falling within the range inclusive of the values defining the range, and each separate value is incorporated in the specification as it were individually recited herein.

As used herein, the term “pharmaceutically acceptable” refers to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable vehicles (e.g., carriers, adjuvants, and/or other excipients) have preferably met the required standards of toxicological and manufacturing testing and/or are included on the Inactive Ingredient Guide prepared by the U.S. Food and Drug administration.

“Pharmaceutically acceptable salts” include, for example, salts with inorganic acids and salts with an organic acid. Examples of salts may include hydrochlorate, phosphate, diphosphate, hydrobromate, sulfate, sulfinate, nitrate, malate, maleate, fumarate, tartrate, succinate, citrate, acetate, lactate, mesylate, p-toluenesulfonate, 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

The term “co-crystal” refers to a crystalline material formed by combining a compound, such as those disclosed herein, and one or more co-crystal formers (i.e., a molecule, ion or atom). In certain instances, co-crystals may have improved properties as compared to the parent form (i.e., the free molecule, zwitterion, etc.) or a salt of the parent compound. Improved properties can be increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like. Methods for making and characterizing co-crystals are known to those of skill in the art.

The term “polymorph” refers to different crystal structures of a crystalline compound. The different polymorphs may result from differences in crystal packing (packing polymorphism) or differences in packing between different conformers of the same molecule (conformational polymorphism).

The term “solvate” refers to an association or complex of one or more solvent molecules and a compound of the disclosure. Examples of solvents that form solvates may include water, isopropanol, ethanol, methanol, dimethylsulfoxide, ethylacetate, acetic acid and ethanolamine.

The term “hydrate” refers to the complex formed by the combining of a compound described herein and water.

The term “prodrug” refers to compounds disclosed herein that include chemical groups which, in vivo, can be converted and/or can be split off from the remainder of the molecule to provide for the active drug, a pharmaceutically acceptable salt thereof, or a biologically active metabolite thereof.

The term “racemates” refers to a mixture of enantiomers.

The terms “stereoisomer” or “stereoisomers” refer to compounds that differ in the chirality of one or more stereocenters. Stereoisomers include enantiomers and diastereomers. The compounds may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

The term “carrier” or “pharmaceutically acceptable carrier” refers to diluents, disintegrants, precipitation inhibitors, surfactants, glidants, binders, lubricants, and other excipients and vehicles with which the compound is administered. Carriers are generally described herein and also in “Remington's Pharmaceutical Sciences” by E. W. Martin. Examples of carriers may include, but are not limited to, aluminum monostearate, aluminum stearate, carboxymethylcellulose, carboxymethylcellulose sodium, crospovidone, glyceryl isostearate, glyceryl monostearate, hydroxyethyl cellulose, hydroxyethyl cellulose, hydroxymethyl cellulose, hydroxyoctacosanyl hydroxystearate, hydroxypropyl cellulose, hydroxypropyl cellulose, hydroxypropyl methylcellulose, lactose, lactose monohydrate, magnesium stearate, mannitol, microcrystalline cellulose, poloxamer 124, poloxamer 181, poloxamer 182, poloxamer 188, poloxamer 237, poloxamer 407, povidone, silicon dioxide, colloidal silicon dioxide, silicone, silicone adhesive 4102, and silicone emulsion. It should be understood, however, that the carriers selected for the pharmaceutical compositions, and the amounts of such carriers in the composition, may vary depending on the method of formulation (e.g., dry granulation formulation, solid dispersion formulation).

The term “diluent” generally refers to a substance used to dilute the compound of interest prior to delivery. Diluents can also serve to stabilize compounds. Examples of diluents may include starch, saccharides, disaccharides, sucrose, lactose, polysaccharides, cellulose, cellulose ethers, hydroxypropyl cellulose, sugar alcohols, xylitol, sorbitol, maltitol, microcrystalline cellulose, calcium or sodium carbonate, lactose, lactose monohydrate, dicalcium phosphate, cellulose, compressible sugars, dibasic calcium phosphate dehydrate, mannitol, microcrystalline cellulose, and tribasic calcium phosphate.

The term “disintegrant” generally refers to a substance which, upon addition to a solid preparation, facilitates its break-up or disintegration after administration and permits the release of an active ingredient as efficiently as possible to allow for its rapid dissolution. Examples of disintegrants may include maize starch, sodium starch glycolate, croscarmellose sodium, crospovidone, microcrystalline cellulose, modified corn starch, sodium carboxymethyl starch, povidone, pregelatinized starch, and alginic acid.

The term “precipitation inhibitors” generally refers to a substance that prevents or inhibits precipitation of the active agent from a supersaturated solution. One example of a precipitation inhibitor includes hydroxypropylmethylcellulose (HPMC).

The term “surfactants” generally refers to a substance that lowers the surface tension between a liquid and a solid that could improve the wetting of the active agent or improve the solubility of the active agent. Examples of surfactants may include poloxamer and sodium lauryl sulfate.

The term “glidant” generally refers to substances used in tablet and capsule formulations to improve flow-properties during tablet compression and to produce an anti-caking effect. Examples of glidants may include colloidal silicon dioxide, talc, fumed silica, starch, starch derivatives, and bentonite.

The term “binder” generally refers to any pharmaceutically acceptable film which can be used to bind together the active and inert components of the carrier together to maintain cohesive and discrete portions. Examples of binders may include hydroxypropylcellulose, hydroxypropylmethylcellulose, povidone, copovidone, and ethyl cellulose.

The term “lubricant” generally refers to a substance that is added to a powder blend to prevent the compacted powder mass from sticking to the equipment during the tableting or encapsulation process. A lubricant can aid the ejection of the tablet form the dies, and can improve powder flow. Examples of lubricants may include magnesium stearate, stearic acid, silica, fats, calcium stearate, polyethylene glycol, sodium stearyl fumarate, or talc; and solubilizers such as fatty acids including lauric acid, oleic acid, and C₈/C₁₀ fatty acid.

“Treating” and “treatment” of a disease include the following: (1) preventing or reducing the risk of developing the disease, i.e., causing the clinical symptoms of the disease not to develop in a subject that may be exposed to or predisposed to the disease but does not yet experience or display symptoms of the disease, (2) inhibiting the disease, i.e., arresting or reducing the development of the disease or its clinical symptoms, and (3) relieving the disease, i.e., causing regression of the disease or its clinical symptoms.

“Subject” and “subjects” refers to humans, domestic animals (e.g., dogs and cats), farm animals (e.g., cattle, horses, sheep, goats and pigs), laboratory animals (e.g., mice, rats, hamsters, guinea pigs, pigs, rabbits, dogs, and monkeys), and the like.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

The terms “effective amount,” “pharmaceutically effective amount,” and “therapeutically effective amount” refer to an amount that may be effective to elicit the desired biological or medical response, including the amount of a compound that, when administered to a subject for treating a disease, is sufficient to effect such treatment for the disease. The effective amount will vary depending on the compound, the disease and its severity and the age, weight, etc., of the subject to be treated. The effective amount can include a range of amounts. A pharmaceutically effective amount includes amounts of an agent which are effective when combined with other agents.

One skilled in the art understands that the compounds disclosed herein may be named or identified using various commonly recognized nomenclature systems and symbols. By way of example, the compounds disclosed herein may be named or identified with common names, systematic or non-systematic names. The nomenclature systems and symbols that are commonly recognized in the art of chemistry include, for example, Chemical Abstract Service (CAS), ChemBioDraw Ultra, and International Union of Pure and Applied Chemistry (IUPAC).

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line (“ [symbol] ”) drawn at the end of a line indicates a specified point of attachment of a group. Unless chemically or structurally required, no directionality or stereochemistry is indicated or implied by the order in which a chemical group is written or named.

Also provided herein are isotopically labeled forms of compounds detailed herein. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated, are provided. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of subjects (e.g., humans). Also provided for are isotopically labeled pharmaceutically acceptable salts, pharmaceutically acceptable esters, pharmaceutically acceptable co-crystals, stereoisomers, tautomers, or polymorphs, as the case may be.

In some embodiments, the compounds disclosed herein may be varied such that from 1 to n hydrogens attached to a carbon atom is/are replaced by deuterium, in which n is the number of hydrogens in the molecule. Such compounds may exhibit increased resistance to metabolism and are thus useful for increasing the half-life of the compound when administered to a mammal. See, for example, Foster, “Deuterium Isotope Effects in Studies of Drug Metabolism”, Trends Pharmacol. Sci. 5(12):524-527 (1984). Such compounds are synthesized by means well known in the art, for example by employing starting materials in which one or more hydrogens have been replaced by deuterium.

Deuterium labeled or substituted therapeutic compounds of the disclosure may have improved DMPK (drug metabolism and pharmacokinetics) properties, relating to absorption, distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compounds provided herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

### Compounds

### Filgotinib

Filgotinib, also known as GLPG0634, is described in U.S. Pat. No. 8,563,545, and has the following structure:

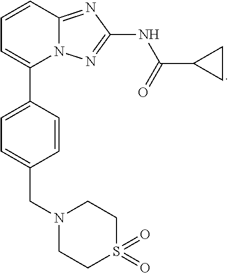 [SMILES: O=C(Nc1nc2cccc(-c3ccc(CN4CCS(=O)(=O)CC4)cc3)n2n1)C1CC1]

Solvates and polymorphs of filgotinib are described in International Application Publication No. WO 2015/117981. A metabolite of filgotinib is described in U.S. Pat. No. 9,284,311. One specific pharmaceutically acceptable salt of filgotinib that may be used in the methods and compositions disclosed herein is the maleate salt of filgotinib.

### Syk Inhibitors

In some embodiments, the Syk inhibitor is entospletinib, which has the following structure:

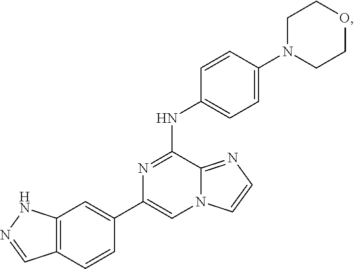 [SMILES: c1cn2cc(-c3ccc4cn[nH]c4c3)nc(Nc3ccc(N4CCOCC4)cc3)c2n1]

or a pharmaceutically acceptable salt, solvate, or polymorph thereof. The chemical name of entospletinib is 6-(1H-indazol-6-yl)-N-(4-morpholinophenyl)imidazo[1,2-a]pyrazin-8-amine. Entospletinib or a pharmaceutically acceptable salt, solvate, or polymorph thereof may be prepared by according to procedures described in U.S. Pat. Nos. 8,748,607 and 8,450,321, and U.S. Patent Application Publication No. 2015/0038505.

In some embodiments, the Syk inhibitor is a compound of Formula I:

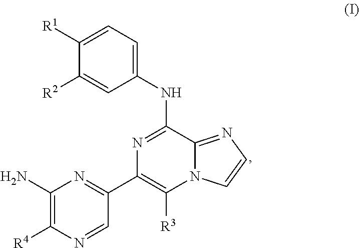

or a pharmaceutically acceptable salt, pharmaceutically acceptable co-crystal, pharmaceutically acceptable ester, stereoisomer, or tautomer thereof,
wherein:

### R¹ is:

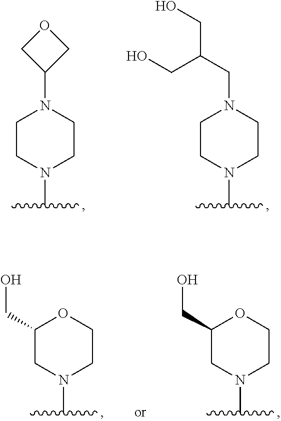 [SMILES: *N1CCN(C2COC2)CC1 ; *N1CCN(CC(CO)CO)CC1 ; *N1CCO[C@@H](CO)C1 ; *N1CCO[C@H](CO)C1]

wherein [symbol] indicates the point of attachment to the remainder of the compound of formula (I),
R² is H or 2-hydroxyethoxy,
R³ is H or methyl, and
R⁴ is H or methyl.

In one embodiment each of R², R³, and R⁴ is H, and R¹ is as defined above.

In one embodiment, R² is H, R³ is methyl, and R⁴ is H, and R¹ is as defined above.

In one embodiment, R² is H, R³ is H, and R⁴ is methyl, and R¹ is as defined above.

In one embodiment, R² is 2-hydroxyethoxy, R³ is methyl, and R⁴ is H, and R¹ is as defined above.

In one embodiment, R² is 2-hydroxyethoxy, R³ is methyl, and R⁴ is H, and R¹ is as defined above.

In one embodiment, R² is 2-hydroxyethoxy, R³ is H, and R⁴ is methyl, and R¹ is as defined above.

In one embodiment, the compound of Formula (I) is selected from Table 1.

TABLE 1

| No. | Structure | Chemical Name |
|---|---|---|
| Ia | 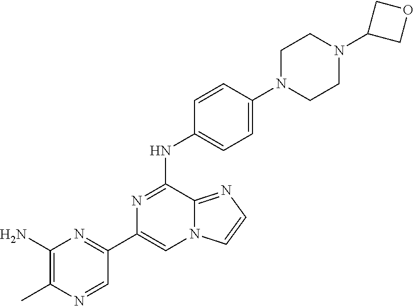 [SMILES: Cc1ncc(-c2cn3ccnc3c(Nc3ccc(N4CCN(C5COC5)CC4)cc3)n2)nc1N] | 6-(6-amino-5-methylpyrazin- 2-yl)-N-(4-(4-(oxetan-3- yl)piperazin-1- yl)phenyl)imidazo[1,2- a]pyrazin-8-amine |
| Ib | 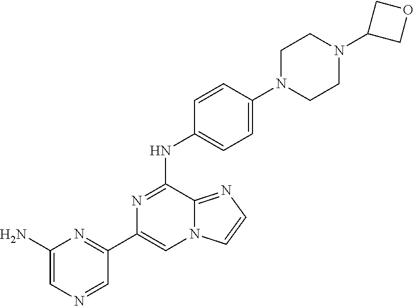 [SMILES: Nc1cncc(-c2cn3ccnc3c(Nc3ccc(N4CCN(C5COC5)CC4)cc3)n2)n1] | 6-(6-aminopyrazin-2-yl)-N- (4-(4-(oxetan-3-yl)piperazin- 1-yl)phenyl)imidazo[1,2- a]pyrazin-8-amine |
| Ic | 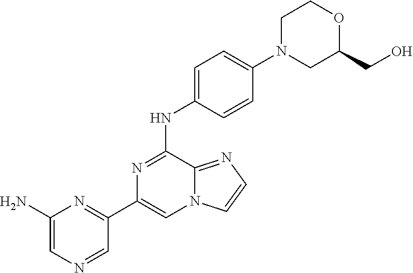 [SMILES: Nc1cncc(-c2cn3ccnc3c(Nc3ccc(N4CCO[C@@H](CO)C4)cc3)n2)n1] | (R)-(4-(4-((6-(6- aminopyrazin-2- yl)imidazo[1,2-a]pyrazin-8- yl)amino)phenyl)morpholin- 2-yl)methanol |
| Id | 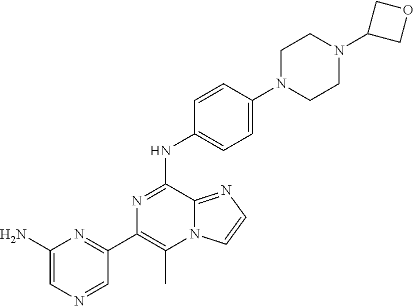 [SMILES: Cc1c(-c2cncc(N)n2)nc(Nc2ccc(N3CCN(C4COC4)CC3)cc2)c2nccn12] | 6-(6-aminopyrazin-2-yl)-5- methyl-N-(4-(4-(oxetan-3- yl)piperazin-1- yl)phenyl)imidazo[1,2- a]pyrazin-8-amine |
| Ie | 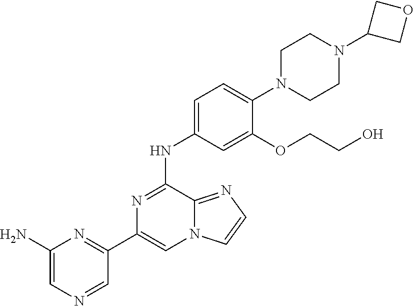 [SMILES: Nc1cncc(-c2cn3ccnc3c(Nc3ccc(N4CCN(C5COC5)CC4)c(OCCO)c3)n2)n1] | 2-(5-((6-(6-aminopyrazin-2- yl)imidazo[1,2-a]pyrazin-8- yl)amino)-2-(4-(oxetan-3- yl)piperazin-1- yl)phenoxy)ethan-1-ol |
| If | 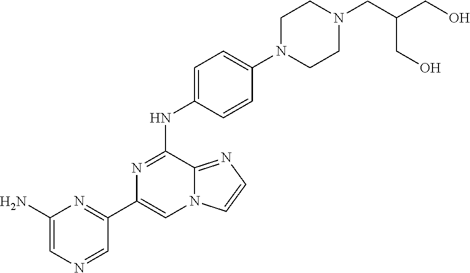 [SMILES: Nc1cncc(-c2cn3ccnc3c(Nc3ccc(N4CCN(CC(CO)CO)CC4)cc3)n2)n1] | 2-((4-(4-((6-(6-aminopyrazin- 2-yl)imidazo[1,2-a]pyrazin-8- yl)amino)phenyl)piperazin-1- yl)methyl)propane-1,3-diol |
| Ig | 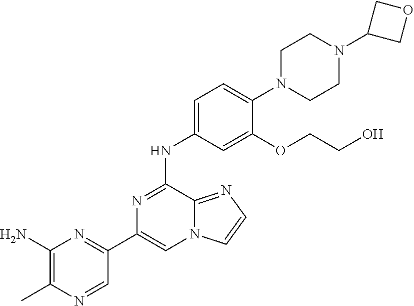 [SMILES: Cc1ncc(-c2cn3ccnc3c(Nc3ccc(N4CCN(C5COC5)CC4)c(OCCO)c3)n2)nc1N] | 2-(5-((6-(6-amino-5- methylpyrazin-2- yl)imidazo[1,2-a]pyrazin-8- yl)amino)-2-(4-(oxetan-3- yl)piperazin-1- yl)phenoxy)ethan-1-ol |

Compounds of Formula (I), or a pharmaceutically acceptable salt, pharmaceutically acceptable co-crystal, pharmaceutically acceptable ester, stereoisomer, or tautomer thereof, are described in U.S. Pat. No. 9,290,050.

Pharmaceutically acceptable salts, esters, stereoisomers, tautomers, prodrugs, solvates, and deuterated forms of the compounds disclosed herein may be used in the methods and compositions disclosed herein.

### Treatment Methods and Uses

Filgotinib and the Syk inhibitors may be used in combination therapies. Accordingly, the present disclosure provides methods for treating inflammatory disorders or cancers in a human in need thereof, comprising administering to the human a therapeutically effective amount of filgotinib or a pharmaceutically acceptable salt, solvate, polymorph, or metabolite thereof, and a therapeutically effective amount of a Syk inhibitor.

In some embodiments, the methods comprising administration of a combination of filgotinib or a pharmaceutically acceptable salt, solvate, polymorph, or metabolite thereof, and a Syk inhibitor, provide synergy. In some embodiments, the amount or dosage of filgotinib, the Syk inhibitor, or both, used in combination, does not exceed the level at which each agent is used individually, e.g., as a monotherapy. In certain embodiments, the amount or dosage of filgotinib, the Syk inhibitor, or both, used in combination, is lower (e.g., at least 20%, at least 30%, at least 40%, or at least 50% lower) than the amount or dosage of each agent used individually, e.g., as a monotherapy. In other embodiments, the amount or dosage of filgotinib, the Syk inhibitor, or both, used in combination that results in treatment of an inflammatory disorder is lower (e.g., at least 20%, at least 30%, at least 40%, or at least 50% lower) than the amount or dosage of each agent used individually, e.g., as a monotherapy.

The present disclosure, in some embodiments, provides a method for treating an inflammatory disorder in a human in need thereof, comprising administering to the human: (i) a therapeutically effective amount of filgotinib or a pharmaceutically acceptable salt, solvate, polymorph, or metabolite thereof; and (ii) a therapeutically effective amount of a Syk inhibitor, wherein the Syk inhibitor is entospletinib or a pharmaceutically acceptable salt, solvate, or polymorph thereof, or a compound of Formula (I) as disclosed herein or a pharmaceutically acceptable salt, pharmaceutically acceptable co-crystal, pharmaceutically acceptable ester, stereoisomer, or tautomer thereof.

In one embodiment, the Syk inhibitor is entospletinib or a pharmaceutically acceptable salt, solvate, or polymorph thereof. In another embodiment, the Syk inhibitor is a compound of Formula (I) or a pharmaceutically acceptable salt, pharmaceutically acceptable co-crystal, pharmaceutically acceptable ester, stereoisomer, or tautomer thereof. In yet another embodiment, the Syk inhibitor is a compound of Formula (I) selected from Table 1, or a pharmaceutically acceptable salt, pharmaceutically acceptable co-crystal, pharmaceutically acceptable ester, stereoisomer, or tautomer thereof.

In some embodiments, the inflammatory disorder is systemic lupus erythematosus, myestenia gravis, rheumatoid arthritis, acute disseminated encephalomyelitis, idiopathic thrombocytopenic purpura, multiple sclerosis, psoriasis, autoimmune hemolytic anemia, asthma, ulcerative colitis, Crohn's disease, irritable bowel disease, chronic obstructive pulmonary disease, Sjogren's syndrome, or graft versus host disease. In one embodiment, the inflammatory disorder is rheumatoid arthritis. In another embodiment, the inflammatory disorder is systemic lupus erythematosus. In another embodiment, the inflammatory disorder is Crohn's disease. In yet another embodiment, the inflammatory disorder is ulcerative colitis.

In some embodiments, a method is provided for treating an inflammatory disorder selected from rheumatoid arthritis, Crohn's disease, ulcerative colitis, osteoarthritis, allergic airway disease, multiple sclerosis, inflammatory bowel disease, systemic lupus erythematosus, psoriasis, Sjogren's syndrome, and graft versus host disease in a human in need thereof, comprising administering to the human: (i) a therapeutically effective amount of filgotinib or a pharmaceutically acceptable salt, solvate, polymorph, or metabolite thereof; and (ii) a therapeutically effective amount of a Syk inhibitor, wherein the Syk inhibitor is entospletinib or a pharmaceutically acceptable salt, solvate, or polymorph thereof, or a compound of Formula (I) as disclosed herein or a pharmaceutically acceptable salt, pharmaceutically acceptable co-crystal, pharmaceutically acceptable ester, stereoisomer, or tautomer thereof.

In one embodiment, the present disclosure provides a method of treating graft versus host disease in a human in need thereof, the method comprising administering to the human: (i) a therapeutically effective amount of filgotinib or a pharmaceutically acceptable salt, solvate, polymorph, or metabolite thereof; and (ii) a therapeutically effective amount of entospletinib or a pharmaceutically acceptable salt, solvate, or polymorph thereof.

In another embodiment, the present disclosure provides a method of treating rheumatoid arthritis in a human in need thereof, the method comprising administering to the human: (i) a therapeutically effective amount of filgotinib or a pharmaceutically acceptable salt, solvate, polymorph, or metabolite thereof; and (ii) a therapeutically effective amount of a compound of Formula (Ib) or a pharmaceutically acceptable salt, pharmaceutically acceptable co-crystal, pharmaceutically acceptable ester, stereoisomer, or tautomer thereof.

In another embodiment, the present disclosure provides a method of treating systemic lupus erythematosus in a human in need thereof, the method comprising administering to the human: (i) a therapeutically effective amount of filgotinib or a pharmaceutically acceptable salt, solvate, polymorph, or metabolite thereof; and (ii) a therapeutically effective amount of a compound of Formula (Ib) or a pharmaceutically acceptable salt, pharmaceutically acceptable co-crystal, pharmaceutically acceptable ester, stereoisomer, or tautomer thereof.

In some embodiments, filgotinib, or a pharmaceutically acceptable salt, solvate, polymorph, or metabolite thereof, is administered intravenously, intramuscularly, parenterally, nasally or orally. In some embodiments, the Syk inhibitor as disclosed herein is administered intravenously, intramuscularly, parenterally, nasally or orally. In some embodiments, filgotinib, or a pharmaceutically acceptable salt, solvate, polymorph, or metabolite thereof, is administered prior, after or concurrently with the Syk inhibitor as disclosed herein.

In some embodiments, the present disclosure provides a method for treating an inflammatory disorder in a human in need thereof, comprising administering to the human a co-formulation of: filgotinib or a pharmaceutically acceptable salt, solvate, polymorph, or metabolite thereof; and a Syk inhibitor. In one embodiment, the Syk inhibitor in the co-formulation is entospletinib or a pharmaceutically acceptable salt, solvate, or polymorph thereof. In another embodiment, the Syk inhibitor in the co-formulation is a compound of Formula (I) or a pharmaceutically acceptable salt, pharmaceutically acceptable co-crystal, pharmaceutically acceptable ester, stereoisomer, or tautomer thereof. In yet another embodiment, the Syk inhibitor in the co-formulation is a compound of Formula (I) selected from Table 1 or a pharmaceutically acceptable salt, pharmaceutically acceptable co-crystal, pharmaceutically acceptable ester, stereoisomer, or tautomer thereof. In still another embodiment, the Syk inhibitor in the co-formulation is a compound of Formula (Ib) or a pharmaceutically acceptable salt, pharmaceutically acceptable co-crystal, pharmaceutically acceptable ester, stereoisomer, or tautomer thereof. In a further embodiment, the co-formulation additionally includes a pharmaceutically acceptable carrier. In an additional embodiment, the co-formulation is administered intravenously, intramuscularly, parenterally, nasally or orally.

In some embodiments, the present disclosure provides use of a composition for the manufacture of a medicament for treating an inflammatory disorder, wherein the composition comprises: (i) filgotinib or a pharmaceutically acceptable salt, solvate, polymorph, or metabolite thereof; and (ii) a Syk inhibitor.

In some embodiments, the present disclosure provides use of a composition for the manufacture of a medicament for treating ulcerative colitis, Crohn's disease, or rheumatoid arthritis, wherein the composition comprises: (i) filgotinib or a pharmaceutically acceptable salt, solvate, polymorph, or metabolite thereof; and (ii) a Syk inhibitor.

### Inflammatory Disorders

In some embodiments, the methods and compositions (or co-formulations) disclosed herein are used in the treatment of an inflammatory disorder. Among the inflammatory disorders are inflammatory bowel disease (IBD) (including Crohn's disease, ulcerative colitis (UC), and indeterminate colitis), collagenous colitis, rheumatoid arthritis, septicemia, sepsis, psoriasis, myestenia gravis, acute disseminated encephalomyelitis, idiopathic thrombocytopenic purpura, Sjogren's syndrome, autoimmune hemolytic anemia, multiple sclerosis, muscular dystrophy, systemic lupus erythematosus, allergy, asthma, chronic obstructive pulmonary disease (COPD), and metabolic disorders characterized by impaired insulin production and glucose intolerance (e.g., Insulin Dependent Diabetes Mellitus (IDDM, also known as type 1 diabetes), and Non-Insulin-Dependent Diabetes Mellitus (NIDDM, also known as type 2 diabetes)).

In some embodiments, the methods and compositions disclosed herein are used to treat a disorder that is causally related or attributable to aberrant activity of JAK, such as disorders related to aberrant activity of JAK1 and/or JAK2. Accordingly, in certain embodiments, the methods and compositions disclosed herein are used to treat inflammatory conditions, autoimmune diseases, proliferative diseases, transplantation rejection, diseases involving impairment of cartilage turnover, congenital cartilage malformations, and diseases associated with hypersecretion of IL6 in mammals including humans.

Accordingly, in one embodiment, are methods and compositions for treating a mammal susceptible to, or afflicted with, an inflammatory condition. In a specific embodiment, the inflammatory condition is selected from rheumatoid arthritis, osteoarthritis, allergic airway disease (e.g., asthma) and inflammatory bowel diseases.

In another embodiment, the methods and compositions disclosed herein are used to treat an autoimmune disease such as COPD, asthma, systemic lupus erythematosus, and type I diabetes mellitus.

In yet another embodiment, the methods and compositions disclosed herein are used to treat transplantation rejection, such as organ transplant rejection.

In additional embodiments, the methods and compositions disclosed herein are used to treat a disease involving impairment of cartilage turnover.

In further embodiments, the methods and compositions disclosed herein are used to treat congenital cartilage malformation.

In various embodiments, the methods and compositions disclosed herein are used to treat a disease associated with hypersecretion of IL6, in particular Castleman's disease or mesangial proliferative glomerulonephritis.

In some embodiments, the disease or condition that may be treated is selected from the group consisting of systemic lupus erythematosus (SLE), myestenia gravis, Goodpasture's syndrome, glomerulonephritis, hemorrhage, pulmonary hemorrhage, atherosclerosis, rheumatoid arthritis (RA), psoriatic arthritis, monoarticular arthritis, osteoarthritis, gouty arthritis, spondylitis, Behçet disease, autoimmune thyroiditis, Reynaud's syndrome, acute disseminated encephalomyelitis, chronic idiopathic thrombocytopenic purpura, multiple sclerosis (MS), Sjogren's syndrome, autoimmune hemolytic anemia, tissue graft rejection, hyperacute rejection of transplanted organs, allograft rejection, graft-versus-host disease, diseases involving leukocyte diapedesis, disease states due to leukocyte dyscrasia and metastasis, granulocyte transfusion-associated syndromes, cytokine-induced toxicity, scleroderma, vasculitis, asthma, psoriasis, inflammatory bowel disease (e.g. chronic inflammatory bowel disease, ulcerative colitis, Crohn's disease, necrotizing enterocolitis), irritable bowel syndrome, dermatomyositis, Addison's disease, Parkinson's disease, Alzheimer's disease, diabetes, type I diabetes mellitus, sepsis, septic shock, endotoxic shock, gram negative sepsis, gram positive sepsis, and toxic shock syndrome, multiple organ injury syndrome secondary to septicemia, trauma, hypovolemic shock, allergic conjunctivitis, vernal conjunctivitis, and thyroid-associated ophthalmopathy, eosinophilic granuloma, eczema, chronic bronchitis, acute respiratory distress syndrome, allergic rhinitis, coryza, hay fever, bronchial asthma, silicosis, pulmonary sarcoidosis, pleurisy, alveolitis, emphysema, pneumonia, bacterial pneumonia, bronchiectasis, and pulmonary oxygen toxicity, reperfusion injury of the myocardium, brain, or extremities, thermal injury, cystic fibrosis, keloid formation or scar tissue formation, fever and myalgias due to infection, and brain or spinal cord injury due to minor trauma, diseases involving leukocyte diapedesis, acute hypersensitivity, delayed hypersensitivity, urticaria, food allergies, skin sunburn, inflammatory pelvic disease, urethritis, uveitis, sinusitis, pneumonitis, encephalitis, meningitis, myocarditis, nephritis, osteomyelitis, myositis, hepatitis, alcoholic hepatitis, gastritis, enteritis, contact dermatitis, atopic dermatitis, gingivitis, appendicitis, pancreatitis, cholocystitis, polycythemia vera, essential thrombocythemia, and polycystic kidney disease.

In some embodiments, the disease is an autoimmune disease. In some embodiments, the autoimmune disease is systemic lupus erythematosus (SLE), myestenia gravis, rheumatoid arthritis (RA), acute disseminated encephalomyelitis, idiopathic thrombocytopenic purpura, multiple sclerosis (MS), Sjoegren's syndrome, psoriasis, autoimmune hemolytic anemia, asthma, ulcerative colitis, Crohn's disease, irritable bowel disease, or chronic obstructive pulmonary disease (COPD). In some embodiments, the disease is excessive or destructive immune reactions, such as asthma, rheumatoid arthritis, multiple sclerosis, chronic obstructive pulmonary disease (COPD), or systemic lupus erythematosus.

### Rheumatoid Arthritis

Rheumatoid Arthritis (RA) is a chronic inflammatory and degenerative joint disease affecting about 1% of the worldwide adult population, with a higher prevalence in women. Although RA can occur at any age, it usually begins between the ages of 40 and 60. In particular, elderly patients are at high risk for adverse events from drug-drug interactions (DDIs) due to chronic disease, physiologic changes associated with aging, and the tendency to use multiple medications. The average older person uses two to six prescription medications and one to three non-prescription medications on a routine basis. The most common mechanism underlying DDI relates to the interplay with cytochrome P450 enzymes (CYP450s), with the inhibition of these enzymes being most often responsible for life-threatening interactions. In addition to these metabolic enzymes, the role drug transporters play in DDI, safety, and effectiveness of drugs has been greatly appreciated in recent years. Transporter-inhibiting drugs, such as filgotinib, can alter the transporter functional activity and/or protein expression, hence causing transporter-specific interactions.

### Inflammatory Bowel Disease

Inflammatory bowel diseases (IBDs) as used herein is a collective term describing inflammatory disorders of the gastrointestinal tract, the most common forms of which are ulcerative colitis and Crohn's disease. Other forms of IBD that can be treated with the presently disclosed compounds, compositions and methods include diversion colitis, ischemic colitis, infectious colitis, chemical colitis, microscopic colitis (including collagenous colitis and lymphocytic colitis), atypical colitis, pseudomembranous colitis, fulminant colitis, autistic enterocolitis, indeterminate colitis, Behçet's disease, gastroduodenal CD, jejunoileitis, ileitis, ileocolitis, Crohn's (granulomatous) colitis, irritable bowel syndrome, mucositis, radiation induced enteritis, short bowel syndrome, celiac disease, stomach ulcers, diverticulitis, pouchitis, proctitis, and chronic diarrhea.

Treating or treatment of IBD includes: (1) preventing or reducing the risk of developing IBD, i.e., causing the clinical symptoms of IBD not to develop in a subject that may be exposed to, or predisposed to, the disease but does not yet experience or display symptoms of IBD, (2) inhibiting the disease, i.e., arresting or reducing the development of IBD, or its clinical symptoms, and (3) relieving IBD, i.e., causing regression of IBD, or its clinical symptoms. Symptoms of IBD refer to detected symptoms including, but not limited to, abdominal pain, diarrhea, rectal bleeding, weight loss, fever, loss of appetite, and other more serious complications, such as dehydration, anemia and malnutrition. A number of such symptoms are subject to quantitative analysis (e.g., weight loss, fever, anemia, etc.). Some symptoms are readily determined from a blood test (e.g., anemia) or a test that detects the presence of blood (e.g., rectal bleeding). Reducing symptoms, such as symptoms of IBD, refers to a qualitative or quantitative reduction in detectable symptoms, including but not limited to a detectable impact on the rate of recovery from disease (e.g., rate of weight gain). The diagnosis is typically determined by way of an endoscopic observation of the mucosa, and pathologic examination of endoscopic biopsy specimens.

The course of IBD varies, and is often associated with intermittent periods of disease remission and disease exacerbation. Various methods have been described for characterizing disease activity and severity of IBD, as well as response to treatment in subjects having IBD. Treatment according to the presently disclosed methods is generally applicable to a subject having IBD of any level or degree of disease activity.

The presently disclosed treatment methods can also be applied at any point in the course of the disease. In certain embodiments, the methods disclosed herein are applied to a subject having IBD during a time period of remission (i.e., inactive disease). In such embodiments, the present methods provide benefit by extending the time period of remission (e.g., extending the period of inactive disease) or by preventing, reducing, or delaying the onset of active disease. In other embodiments, the methods disclosed herein may be applied to a subject having IBD during a period of active disease. Such methods provide benefit by reducing the duration of the period of active disease, reducing or ameliorating one or more symptoms of IBD, or treating IBD.

Measures for determining efficacy of treatment of IBD in clinical practice have been described and include, for example, the following: symptom control; fistula closure; extent of corticosteroid therapy required; and, improvement in quality of life. Heath-related quality of life (HRQL) can be assessed using the Inflammatory Bowel Disease Questionnaire (IBDQ), which is extensively used in clinical practice to assess quality of life in a subject with IBD. Improvements in any of the foregoing response criteria are specifically provided by the methods of the present disclosure.

As indicated above, ulcerative colitis (UC) is one of the two major IBDs, characterized by diffuse mucosal inflammation, and associated ulceration, of the colon. The chronic course of UC includes intermittent disease exacerbations followed by periods of remission. Many patients experience insufficient response to agents such as anti-TNFα targeted therapeutics and continue to suffer from disease-related symptoms. Patients with UC have a significantly elevated risk of colon cancer after 8-10 years of disease activity.

Crohn's disease (CD) is a chronic inflammatory disorder of the gastrointestinal tract defined by relapsing and remitting episodes, with progression to complications such as fistula formation, abscesses, or strictures. Extraintestinal manifestations such as uveitis, arthritis, skin lesions, and kidney stones occur in upwards of 40% of patients. The treatment paradigm for mild-to-moderate Crohn's has been antibiotics such as ciprofloxacin and flagyl, 5-ASAs, budesonide, or systemic corticosteroids, however, the long-term side effects of systemic steroids greatly dampens their utility. Patients with mild-to-moderate disease who fail these first line therapies are often placed on the on azathioprine remain in remission at one-year. For patients who fail azathioprine or those with more severe disease, TNF-α blockade with agents such as infliximab remain the last option. As opposed to UC where surgical resection is curative, such therapy is more difficult for Crohn's patients for two reasons: 1) disease is diffuse throughout the GI tract and in instances of isolated disease (e.g., terminal ileum), resection is frequently associated with recurrent disease at the site of the resection 2) since the disease is transmural, surgical resection places patients at risk for future stricture and/or fistula development.

Inflammatory bowel disease (IBD) therapeutics can modulate disease by preventing recruitment and access of inflammatory cells to the disease site, preventing activation of cells at the disease site, and/or inhibiting the downstream effects of cell activation.

### Cancer

Provided herein are methods for treating cancers in a human in need thereof, comprising administering to the human a therapeutically effective amount of filgotinib or a pharmaceutically acceptable salt, solvate, polymorph, or metabolite thereof, and a therapeutically effective amount of a Syk inhibitor.

In some embodiments, the cancer is a hematologic malignancy. In certain embodiments, the cancer is a leukemia. In one embodiment, the leukemia is chronic lymphocytic leukemia (CLL). In certain embodiments, the cancer is a lymphoma. In one embodiment, the lymphoma is non-Hodgkin's lymphoma (NHL). In one variation, the NHL is diffuse large B-cell lymphoma (DLBCL), mantle cell lymphoma (MCL), follicular lymphoma (FL), small lymphocytic lymphoma (SLL), lymphoplasmacytic lymphoma (LPL), and/or marginal zone lymphoma (MZL). Thus, it is understood that in one aspect the subject is a human who has a hematologic malignancy, such as leukemia or lymphoma.

In certain embodiments, the cancer is selected from the group consisting of acute lymphocytic leukemia (ALL), acute myeloid leukemia (AML), chronic lymphocytic leukemia (CLL), small lymphocytic lymphoma (SLL), myelodysplastic syndrome (MDS), myeloproliferative disease (MPD), chronic myeloid leukemia (CML), multiple myeloma (MM), non-Hodgkin's lymphoma (NHL), indolent non-Hodgkin's lymphoma (iNHL), refractory iNHL, mantle cell lymphoma (MCL), follicular lymphoma (FL), Waldestrom's macroglobulinemia (WM), T-cell lymphoma, B-cell lymphoma, diffuse large B-cell lymphoma (DLBCL), lymphoplasmacytic lymphoma (LPL), and marginal zone lymphoma (MZL).

In some embodiments, the cancer is a solid tumor cancer (or solid cancer tumor). In certain embodiments the cancer is a solid tumor and expresses spleen tyrosine kinase (Syk) activity. In other embodiments, the solid tumor cancer is selected from the group consisting of pancreatic, lung, colorectal cancer, ovarian, and hepatocellular.

### Subject

The human in need thereof may be an individual who has or is suspected of having an inflammatory disorder. In some embodiments, the human is at risk of developing an inflammatory disorder (e.g., a human who is genetically or otherwise predisposed to developing an inflammatory disorder) and who has or has not been diagnosed with the inflammatory disorder. As used herein, an “at risk” subject is a subject who is at risk of developing an inflammatory disorder. The subject may or may not have detectable disease, and may or may not have displayed detectable disease prior to the treatment methods described herein. An at risk subject may have one or more so-called risk factors, which are measurable parameters that correlate with development of an inflammatory disorder, such as described herein. A subject having one or more of these risk factors has a higher probability of developing an inflammatory disorder than an individual without these risk factor(s).

These risk factors may include, for example, age, sex, race, diet, history of previous disease, presence of precursor disease, genetic (e.g., hereditary) considerations, and environmental exposure. In some embodiments, a human at risk for an inflammatory disorder includes, for example, a human whose relatives have experienced this disease, and those whose risk is determined by analysis of genetic or biochemical markers. Prior history of having an inflammatory disorder may also be a risk factor for instances of recurrence thereof.

In some embodiments, provided herein is a method for treating a human who exhibits one or more symptoms associated with an inflammatory disorder. The human may be at various stages (e.g., an early stage, an advanced stage, etc.) of the inflammatory disorder.

In some embodiments, provided herein is a method for treating a human who is undergoing one or more standard therapies for treating an inflammatory disorder. Thus, in some foregoing embodiments, the combination of filgotinib or a pharmaceutically acceptable salt, solvate, polymorph, or metabolite thereof, and a Syk inhibitor, may be administered before, during, or after administration of such standard therapies.

### Kits

Compositions (including, for example, formulations, co-formulations and unit dosages) comprising filgotinib or a pharmaceutically acceptable salt, solvate, polymorph, or metabolite thereof, and a Syk inhibitor, can be prepared and placed in an appropriate container, and labeled for treatment of an indicated condition. Accordingly, provided herein is also an article of manufacture, such as a container comprising a unit dosage form of filgotinib or a pharmaceutically acceptable salt, solvate, polymorph, or metabolite thereof, and a unit dosage form of a Syk inhibitor, and a label containing instructions for use of the compounds. In some embodiments, the article of manufacture is a container comprising (i) a unit dosage form of filgotinib or a pharmaceutically acceptable salt, solvate, polymorph, or metabolite thereof, and one or more pharmaceutically acceptable carriers, adjuvants or excipients; and (ii) a unit dosage form of a Syk inhibitor, and one or more pharmaceutically acceptable carriers, adjuvants or excipients. In one embodiment, the unit dosage form for both filgotinib or a pharmaceutically acceptable salt, solvate, polymorph, or metabolite thereof, and the Syk inhibitor, is a tablet.

In some embodiments, the article of manufacture may be a bottle, vial, ampoule, single-use disposable applicator, or the like, containing the pharmaceutical composition provided in the present disclosure. The container may be formed from a variety of materials, such as glass or plastic and in one aspect also contains a label on, or associated with, the container which indicates directions for use in the treatment of a medical condition. It should be understood that the active ingredient may be packaged in any material capable of improving chemical and physical stability, such as an aluminum foil bag. In some embodiments, diseases or medical conditions indicated on the label can include, for example, treatment of an inflammatory disorder.

Kits also are contemplated. For example, a kit can comprise unit dosage forms of filgotinib or a pharmaceutically acceptable salt, solvate, polymorph, or metabolite thereof, and unit dosage forms of a Syk inhibitor, and a package insert containing instructions for use of the composition in treatment of a medical condition, such as an inflammatory disorder.

In some embodiments, the kits comprise (i) a unit dosage form of filgotinib or a pharmaceutically acceptable salt, solvate, polymorph, or metabolite thereof, and one or more pharmaceutically acceptable carriers, adjuvants or excipients; and (ii) a unit dosage form of a Syk inhibitor, and one or more pharmaceutically acceptable carriers, adjuvants or excipients. In one embodiment, the unit dosage form for both filgotinib or a pharmaceutically acceptable salt, solvate, polymorph, or metabolite thereof, and the Syk inhibitor, is a tablet. The instructions for use in the kit may be for treating an inflammatory disorder, as further described herein.

In some embodiments, the present disclosure provides a kit comprising: (i) a pharmaceutical composition comprising filgotinib or a pharmaceutically acceptable salt, solvate, polymorph, or metabolite thereof; (ii) a pharmaceutical composition comprising entospletinib or a pharmaceutically acceptable salt, solvate, or polymorph thereof, or a compound of Formula (I) or a pharmaceutically acceptable salt, pharmaceutically acceptable co-crystal, pharmaceutically acceptable ester, stereoisomer, or tautomer thereof; and (iii) instructions for use of (i) and (ii) in treating an inflammatory disorder or cancer.

In one embodiment, the present disclosure provides a kit comprising: (i) a pharmaceutical composition comprising filgotinib or a pharmaceutically acceptable salt, solvate, polymorph, or metabolite thereof; (ii) a pharmaceutical composition comprising entospletinib or a pharmaceutically acceptable salt, solvate, or polymorph thereof; and (iii) instructions for use of (i) and (ii) in treating graft versus host disease.

In another embodiment, the present disclosure provides a kit comprising: (i) a pharmaceutical composition comprising filgotinib or a pharmaceutically acceptable salt, solvate, polymorph, or metabolite thereof; (ii) a pharmaceutical composition comprising a compound of Formula (Ib) or a pharmaceutically acceptable salt, pharmaceutically acceptable co-crystal, pharmaceutically acceptable ester, stereoisomer, or tautomer thereof; and (iii) instructions for use of (i) and (ii) in treating an rheumatoid arthritis or systemic lupus erythematosus.

### Pharmaceutical Compositions and Modes of Administration

Provided herein are pharmaceutical compositions and co-formulations comprising filgotinib or a pharmaceutically acceptable salt, solvate, polymorph, or metabolite thereof, and a Syk inhibitor. In various embodiments, the term “co-formulation” may refer to a composition comprising at least two active ingredients, such as filgotinib or a pharmaceutically acceptable salt, solvate, polymorph, or metabolite thereof, and a Syk inhibitor.

In some embodiments, the present disclosure provides a pharmaceutical composition comprising: (i) filgotinib or a pharmaceutically acceptable salt, solvate, polymorph, or metabolite thereof; (ii) entospletinib or a pharmaceutically acceptable salt, solvate, or polymorph thereof, or a compound of Formula (I) or a pharmaceutically acceptable salt, pharmaceutically acceptable co-crystal, pharmaceutically acceptable ester, stereoisomer, or tautomer thereof; and (iii) a pharmaceutically acceptable carrier. In various embodiments, such a pharmaceutical composition exhibits synergy in treating an inflammatory disorder or cancer.

In one embodiment, the present disclosure provides a pharmaceutical composition comprising: (i) filgotinib or a pharmaceutically acceptable salt, solvate, polymorph, or metabolite thereof; (ii) entospletinib or a pharmaceutically acceptable salt, solvate, or polymorph thereof; and (iii) a pharmaceutically acceptable carrier. In various embodiments, such a pharmaceutical composition exhibits synergy in treating graft versus host disease.

In another embodiment, the present disclosure provides a pharmaceutical composition comprising: (i) filgotinib or a pharmaceutically acceptable salt, solvate, polymorph, or metabolite thereof; (ii) a compound of Formula (Ib) or a pharmaceutically acceptable salt, pharmaceutically acceptable co-crystal, pharmaceutically acceptable ester, stereoisomer, or tautomer thereof; and (iii) a pharmaceutically acceptable carrier. In various embodiments, such a pharmaceutical composition exhibits synergy in treating rheumatoid arthritis or systemic lupus erythematosus.

In some embodiments, the present disclosure provides a co-formulation comprising: (i) filgotinib or a pharmaceutically acceptable salt, solvate, polymorph, or metabolite thereof; (ii) entospletinib or a pharmaceutically acceptable salt, solvate, or polymorph thereof, or a compound of Formula (I) as disclosed herein or a pharmaceutically acceptable salt, pharmaceutically acceptable co-crystal, pharmaceutically acceptable ester, stereoisomer, or tautomer thereof; and (iii) a pharmaceutically acceptable carrier. In various embodiments, such a co-formulation exhibits synergy in treating an inflammatory disorder or cancer.

In one embodiment, the present disclosure provides a co-formulation comprising: (i) filgotinib or a pharmaceutically acceptable salt, solvate, polymorph, or metabolite thereof; (ii) entospletinib or a pharmaceutically acceptable salt, solvate, or polymorph thereof; and (iii) a pharmaceutically acceptable carrier. In various embodiments, such a co-formulation exhibits synergy in treating graft versus host disease.

In another embodiment, the present disclosure provides a co-formulation comprising: (i) filgotinib or a pharmaceutically acceptable salt, solvate, polymorph, or metabolite thereof; (ii) a compound of Formula (Ib) or a pharmaceutically acceptable salt, pharmaceutically acceptable co-crystal, pharmaceutically acceptable ester, stereoisomer, or tautomer thereof; and (iii) a pharmaceutically acceptable carrier. In various embodiments, such a co-formulation exhibits synergy in treating rheumatoid arthritis or systemic lupus erythematosus.

In additional embodiments, the present disclosure provides combination therapy for treating an inflammatory disorder, wherein separate compositions of filgotinib or a pharmaceutically acceptable salt, solvate, polymorph, or metabolite thereof, and a Syk inhibitor are used. For example, a composition comprising filgotinib or a pharmaceutically acceptable salt, solvate, polymorph, or metabolite thereof, and a composition comprising a Syk inhibitor as disclosed herein (e.g., entospletinib or a pharmaceutically acceptable salt, solvate, or polymorph thereof, or a compound of Formula (I) or a pharmaceutically acceptable salt, pharmaceutically acceptable co-crystal, pharmaceutically acceptable ester, stereoisomer, or tautomer thereof) are used separately for the combination therapy.

The pharmaceutical compositions and/or co-formulations disclosed herein may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

In some embodiments, the compositions and/or co-formulations disclosed herein may be administered orally. Oral administration may be via, for example, capsule or enteric coated tablets. In making the pharmaceutical compositions and/or co-formulations described herein, the active ingredient(s) is(are) usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions and/or co-formulations disclosed herein can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

In the preparation of solid pharmaceutical compositions and co-formulations such as tablets, the principal active ingredient(s) may be mixed with a pharmaceutical excipient to form a solid preformulation composition containing a homogeneous mixture of filgotinib or a pharmaceutically acceptable salt, solvate, polymorph, or metabolite thereof, and/or a Syk inhibitor as disclosed herein. When referring to these preformulation compositions as homogeneous, the active ingredient(s) may be dispersed evenly throughout the composition so that the composition may be readily subdivided into equally effective unit dosage forms such as tablets, pills and capsules.

The tablets or pills comprising at least one of the compounds described herein may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

In some embodiments, the pharmaceutical compositions and co-formulations disclosed herein can be formulated so as to provide quick, sustained or delayed release of the active ingredient(s) after administration to the subject by employing procedures known in the art. Controlled release drug delivery systems for oral administration include osmotic pump systems and dissolutional systems containing polymer-coated reservoirs or drug-polymer matrix formulations.

Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. Such patches may be constructed for continuous, pulsatile, or on demand delivery of pharmaceutical agents.

### Dosing

The dosing regimen of filgotinib or a pharmaceutically acceptable salt, solvate, polymorph, or metabolite thereof, and a Syk inhibitor disclosed herein (e.g., entospletinib or a pharmaceutically acceptable salt, solvate, or polymorph thereof, or a compound of Formula (I) or a pharmaceutically acceptable salt, pharmaceutically acceptable co-crystal, pharmaceutically acceptable ester, stereoisomer, or tautomer thereof) in the methods provided herein may vary depending upon the indication, route of administration, and severity of the condition. For instance, depending on the route of administration, a suitable dose can be calculated according to body weight, body surface area, or organ size. The final dosing regimen is determined by the attending physician in view of good medical practice, considering various factors that modify the action of drugs, e.g., the specific activity of the compound, the identity and severity of the disease state, the responsiveness of the subject, the age, condition, body weight, sex, and diet of the subject, and the severity of any infection. Additional factors that can be taken into account include time and frequency of administration, drug combinations, reaction sensitivities, and tolerance/response to therapy. Further refinement of the doses appropriate for treatment involving any of the formulations mentioned herein is done routinely by the skilled practitioner without undue experimentation, especially in light of the dosing information and assays disclosed, as well as the pharmacokinetic data observed in human clinical trials. Appropriate doses can be ascertained through use of established assays for determining concentration of the agent in a body fluid or other sample together with dose response data.

Accordingly, the formulation, route of administration, dosage and dosing frequency of filgotinib or a pharmaceutically acceptable salt, solvate, polymorph, or metabolite thereof, entospletinib or a pharmaceutically acceptable salt, solvate, or polymorph thereof, or a compound of Formula (I) or a pharmaceutically acceptable salt, pharmaceutically acceptable co-crystal, pharmaceutically acceptable ester, stereoisomer, or tautomer thereof, may be based on one or more factors disclosed herein, and tailored to the individual subject, the nature of the condition to be treated in the subject, and generally, the judgment of the attending practitioner.

In some embodiments, a therapeutically effective amount or a pharmaceutically effective amount refers to an amount that is sufficient to effect treatment, when administered to a subject (e.g., a human) in need of such treatment. In one embodiment, a therapeutically effective amount of filgotinib, or a pharmaceutically acceptable salt, solvate, polymorph, or metabolite thereof, is an amount sufficient to modulate JAK expression, and thereby treat a human suffering an indication, or to ameliorate or alleviate the existing symptoms of the indication. In another embodiment, a therapeutically effective amount of entospletinib or a pharmaceutically acceptable salt, solvate, or polymorph thereof, or a therapeutically effective amount of a compound of Formula (I) or a pharmaceutically acceptable salt, pharmaceutically acceptable co-crystal, pharmaceutically acceptable ester, stereoisomer, or tautomer thereof, is an amount sufficient to modulate activity of anti-apoptotic Syk proteins, and thereby treat a human suffering an indication, or to ameliorate or alleviate the existing symptoms of the indication.

In some embodiments, the therapeutically effective amount of any of the compounds disclosed herein may be determined based on data obtained from assays known in the art, including for example, an apoptosis assay.

The therapeutically effective amount of any of the compounds disclosed herein may be provided in a single dose or multiple doses to achieve the desired treatment endpoint. As used herein, “dose” refers to the total amount of an active ingredient (e.g., filgotinib or a pharmaceutically acceptable salt, solvate, polymorph, or metabolite thereof; or entospletinib or a pharmaceutically acceptable salt, solvate, or polymorph thereof; or a compound of Formula (I) as disclosed herein or a pharmaceutically acceptable salt, pharmaceutically acceptable co-crystal, pharmaceutically acceptable ester, stereoisomer, or tautomer thereof) to be taken each time by a subject (e.g., a human).

In some embodiments, the compounds disclosed herein may be provided in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. For instance, in some embodiments, each dosage unit, for oral administration, contains from about 10 mg to about 1000 mg of a compound disclosed herein, for example from about 50 mg to about 500 mg, for example about 50 mg, about 75 mg, about 100 mg, about 150 mg, about 200 mg, about 250 mg, or about 300 mg. In other embodiments, for parenteral administration, each dosage unit contains from 0.1 to 700 mg of a compound disclosed herein.

The dose of any of the compounds disclosed herein may be administered once daily (QD), twice daily (BID), three times daily, four times daily, or more than four times daily using any suitable mode described herein (e.g., oral administration). In some embodiments, the dose of any of the compounds disclosed herein is administered once daily. In some embodiments, the dose of any of the compounds disclosed herein is administered twice daily.

Moreover, administration or treatment with the compounds disclosed herein may be continued for a number of days; for example, treatment may continue for at least 7 days, 14 days, or 28 days, for one cycle of treatment. Treatment cycles are well known, and are frequently alternated with resting periods of about 1 to 28 days, commonly about 7 days or about 14 days, between cycles. The treatment cycles, in other embodiments, may also be continuous.

In some embodiments, filgotinib, or a pharmaceutically acceptable salt, solvate, polymorph, or metabolite thereof, is administered to a human at a dose between 40 mg and 1200 mg, between 40 mg and 800 mg, between 40 mg and 600 mg, or between 40 mg and 400 mg.

In some embodiments, the therapeutically effective amount of filgotinib, or a pharmaceutically acceptable salt, solvate, polymorph, or metabolite thereof, is administered to a human at a dose of from about 1 mg to about 200 mg, about 10 mg to about 200 mg, about 100 mg to about 200 mg, about 50 mg to about 175 mg, about 20 mg to about 160 mg, about 20 mg to about 150 mg, about 10 mg to about 100 mg, or about 75 mg to about 100 mg. In some embodiments, filgotinib, or a pharmaceutically acceptable salt, solvate, polymorph, or metabolite thereof, is administered to a human at a dose between about 50 mg to about 200 mg.

In some embodiments, individual doses of filgotinib, or a pharmaceutically acceptable salt, solvate, polymorph, or metabolite thereof, that may be administered to a human in need thereof may include individual doses of 1 mg, 5 mg, 10 mg, 20 mg, 30 mg, 40 mg, 50 mg, 60 mg, 70 mg, 75 mg, 80 mg, 900 mg, 100 mg, 110 mg, 120 mg, 130 mg, 140 mg, 150 mg, 160 mg, 170 mg, 175 mg, or 200 mg. In additional embodiments, filgotinib, or a pharmaceutically acceptable salt, solvate, polymorph, or metabolite thereof, may be administered to a human at an individual dose of about 300 mg, about 400 mg, about 500 mg, about 600 mg, about 700 mg, or about 800 mg. In some embodiments, filgotinib, or a pharmaceutically acceptable salt, solvate, polymorph, or metabolite thereof, is administered to a human at a dose of about 100 mg. In some embodiments, filgotinib, or a pharmaceutically acceptable salt, solvate, polymorph, or metabolite thereof, is administered to a human at a dose of about 200 mg.

The doses of filgotinib, or a pharmaceutically acceptable salt, solvate, polymorph, or metabolite thereof, disclosed herein may be administered once daily, twice daily, three times daily, or four or more times daily. For example, in some embodiments, the dosage of filgotinib, or a pharmaceutically acceptable salt, solvate, polymorph, or metabolite thereof, is about 50 mg to about 200 mg once, twice, three times, four times, or more than four times daily. In some embodiments, the dosage of filgotinib, or a pharmaceutically acceptable salt, solvate, polymorph, or metabolite thereof, is about 50 mg to about 200 mg once daily. In some embodiments the dosage of filgotinib, or a pharmaceutically acceptable salt, solvate, polymorph, or metabolite thereof, is about 50 mg, about 75 mg, about 100 mg, about 125 mg, about 175 mg, or about 200 mg once daily. In some embodiments, the dosage of filgotinib, or a pharmaceutically acceptable salt, solvate, polymorph, or metabolite thereof, is about 100 mg once daily. In some embodiments, the dosage of filgotinib, or a pharmaceutically acceptable salt, solvate, polymorph, or metabolite thereof, is about 200 mg once daily.

In certain embodiments, filgotinib, or a pharmaceutically acceptable salt, solvate, polymorph, or metabolite thereof, is formulated as a capsule or a tablet.

In some embodiments, the therapeutically effective amount of filgotinib, or a pharmaceutically acceptable salt, solvate, polymorph, or metabolite thereof, may be an amount sufficient to decrease a symptom of a disease or condition responsive to inhibition of JAK activity. For instance, in certain embodiments, filgotinib, or a pharmaceutically acceptable salt, solvate, polymorph, or metabolite thereof, is administered to a human at a dose resulting in about 50%, about 55%, about 60%, about 65%, about 70%, about 75%, about 80%, about 90%, about 95%, or about 99% JAK target inhibition.

In some embodiments, the Syk inhibitors disclosed herein, such as entospletinib or a pharmaceutically acceptable salt, solvate, or polymorph thereof, or a compound of Formula (I) or a pharmaceutically acceptable salt, pharmaceutically acceptable co-crystal, pharmaceutically acceptable ester, stereoisomer, or tautomer thereof, are administered to a human at a dose from about 1 mg to about 5000 mg, about 1 mg to about 4000 mg, about 1 mg to about 3000 mg, about 1 mg to about 2000 mg, about 1 mg to about 1000 mg, about 50 mg to about 1000 mg, about 100 mg to about 1000 mg, about 150 mg to about 1000 mg, about 200 mg to about 1000 mg, about 250 mg to about 1000 mg, about 300 mg to about 1000 mg, about 350 mg to about 1000 mg, about 400 mg to about 1000 mg, about 450 mg to about 1000 mg, about 500 mg to about 1000 mg, about 550 mg to about 1000 mg, about 600 mg to about 1000 mg, about 650 mg to about 1000 mg, about 700 mg to about 1000 mg, about 750 mg to about 1000 mg, about 800 mg to about 1000 mg, about 850 mg to about 1000 mg, about 900 mg to about 1000 mg, about 950 mg to about 1000 mg, about 1 mg to about 750 mg, about 50 mg to about 750 mg, about 100 mg to about 750 mg, about 150 mg to about 750 mg, about 200 mg to about 750 mg, about 250 mg to about 750 mg, about 300 mg to about 750 mg, about 350 mg to about 750 mg, about 400 mg to about 750 mg, about 450 mg to about 750 mg, about 500 mg to about 750 mg, about 550 mg to about 750 mg, about 600 mg to about 750 mg, about 650 mg to about 750 mg, about 700 mg to about 750 mg, about 1 mg to about 500 mg, about 50 mg to about 500 mg, about 100 mg to about 500 mg, about 150 mg to about 500 mg, about 200 mg to about 500 mg, about 250 mg to about 500 mg, about 300 mg to about 500 mg, about 350 mg to about 500 mg, about 400 mg to about 500 mg, about 450 mg to about 500 mg, about 1 mg to about 400 mg, about 50 mg to about 400 mg, about 100 mg to about 400 mg, about 150 mg to about 400 mg, about 200 mg to about 400 mg, about 250 mg to about 400 mg, about 300 mg to about 400 mg, about 350 mg to about 400 mg, about 1 mg to about 300 mg, about 50 mg to about 300 mg, about 100 mg to about 300 mg, about 150 mg to about 300 mg, about 200 mg to about 300 mg, about 250 mg to about 300 mg, about 1 mg to about 250 mg, about 50 mg to about 250 mg, about 100 mg to about 250 mg, about 150 mg to about 250 mg, bout 200 mg to about 250 mg, about 1 mg to about 225 mg, about 25 mg to about 225 mg, about 50 mg to about 225 mg, about 75 mg to about 225 mg, about 100 mg to about 225 mg, about 125 mg to about 225 mg, about 150 mg to about 225 mg, about 175 mg to about 225 mg, about 200 mg to about 225 mg, about 1 mg to about 200 mg, about 25 mg to about 200 mg, about 50 mg to about 200 mg, about 75 mg to about 200 mg, about 100 mg to about 200 mg, about 125 mg to about 200 mg, about 150 mg to about 200 mg, about 175 mg to about 200 mg, about 1 mg to about 175 mg, about 25 mg to about 175 mg, about 50 mg to about 175 mg, about 75 mg to about 175 mg, about 100 mg to about 175 mg, about 125 mg to about 175 mg, about 150 mg to about 175 mg, about 1 mg to about 150 mg, about 25 mg to about 150 mg, about 50 mg to about 150 mg, about 75 mg to about 150 mg, about 100 mg to about 150 mg, about 125 mg to about 150 mg, about 1 mg to about 125 mg, about 25 mg to about 125 mg, about 50 mg to about 125 mg, about 75 mg to about 125 mg, about 100 mg to about 125 mg, about 1 mg to about 100 mg, about 25 mg to about 100 mg, about 50 mg to about 100 mg, or about 75 mg to about 100 mg.

In some embodiments, the Syk inhibitors disclosed herein, such as entospletinib or a pharmaceutically acceptable salt, solvate, or polymorph thereof, or a compound of Formula (I) or a pharmaceutically acceptable salt, pharmaceutically acceptable co-crystal, pharmaceutically acceptable ester, stereoisomer, or tautomer thereof, are administered to a human at a dose of about 1 mg, about 2 mg, about 5 mg, about 10 mg, about 15 mg, about 20 mg, about 25 mg, about 30 mg, about 35 mg, about 40 mg, about 45 mg, about 50 mg, about 60 mg, about 65 mg, about 70 mg, about 75 mg, about 100 mg, about 125 mg, about 150 mg, about 175 mg, about 180 mg, about 190 mg, about 200 mg, about 225 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1200 mg, about 1400 mg, about 1600 mg, about 1800 mg, about 2000 mg, about 2200 mg, about 2400 mg, about 2600 mg, about 2800 mg, about 3000 mg, about 3200 mg, about 3400 mg, about 3600 mg, about 3800 mg, about 4000 mg, about 4200 mg, about 4400 mg, about 4600 mg, about 4800 mg, or about 5000 mg.

In some embodiments, entospletinib or a pharmaceutically acceptable salt, solvate, or polymorph thereof, is administered to a human at a dose of about 200 mg. In some embodiments, entospletinib or a pharmaceutically acceptable salt, solvate, or polymorph thereof, is administered to a human at a dose of about 400 mg. In some embodiments, entospletinib or a pharmaceutically acceptable salt, solvate, or polymorph thereof, is administered to a human at a dose of about 800 mg.

In some embodiments, the compound of Formula (Ib), or a pharmaceutically acceptable salt, pharmaceutically acceptable co-crystal, pharmaceutically acceptable ester, stereoisomer, or tautomer thereof, is administered to a human at a dose of about 15 mg. In some embodiments, the compound of Formula (Ib), or a pharmaceutically acceptable salt, pharmaceutically acceptable co-crystal, pharmaceutically acceptable ester, stereoisomer, or tautomer thereof, is administered to a human at a dose of about 30 mg. In some embodiments, the compound of Formula (Ib), or a pharmaceutically acceptable salt, pharmaceutically acceptable co-crystal, pharmaceutically acceptable ester, stereoisomer, or tautomer thereof, is administered to a human at a dose of about 50 mg.

The doses of a Syk inhibitor as disclosed herein may be administered once daily, twice daily, three times daily, or four or more times daily. For instance, in some embodiments, about 50 mg to 800 mg of a Syk inhibitor as disclosed herein, or a pharmaceutically acceptable salt, pharmaceutically acceptable co-crystal, pharmaceutically acceptable ester, stereoisomer, or tautomer thereof, is administered to a subject once, twice, three times, or four times daily. In some embodiments, individual doses of a Syk inhibitor as disclosed herein that may be administered once, twice, three times, or four times daily to a human in need thereof may include 10 mg, 20 mg, 40 mg, 50 mg, 60 mg, 75 mg, 80, mg, 90 mg, 100 mg, 120 mg, 150 mg, 175 mg, 250 mg, 350 mg, 450 mg, 550 mg, 600 mg, 650 mg, 700 mg, 750 mg, and 800 mg.

In one embodiment, about 100 mg of a Syk inhibitor as disclosed herein is administered to a subject once, twice, three times, or four times daily. In another embodiment, about 200 mg of a compound of a Syk inhibitor as disclosed herein is administered to a subject once, twice, three time, or four times daily. In yet another embodiment, about 300 mg of a Syk inhibitor as disclosed herein is administered to a subject once, twice, three times, or four times daily. In a further embodiment, about 400 mg of a Syk inhibitor is administered to a subject once, twice, three times, or four times daily.

In certain embodiments, a Syk inhibitor as disclosed herein is formulated as a capsule or a tablet. In certain embodiments, the capsule or tablet comprises about 10 mg, about 25 mg, about 50 mg, about 75 mg, about 100 mg, about 150 mg, about 200 mg, about 250 mg, about 300 mg, about 350 mg, about 400 mg, about 450 mg, or about 500 mg of the Syk inhibitor. In certain embodiments, the capsule or tablet comprises from about 50 mg to about 500 mg of the Syk inhibitor. In certain embodiments, the capsule or tablet comprises about 50 mg, about 100 mg, about 150 mg, about 200 mg, about 250, about 300, about 350, about 400, about 450, about 500 mg of the Syk inhibitor.

In certain embodiments, the therapeutically effective amount of a Syk inhibitor as disclosed herein (e.g., entospletinib or a pharmaceutically acceptable salt, solvate, or polymorph thereof, or a compound of Formula (I) or a pharmaceutically acceptable salt, pharmaceutically acceptable co-crystal, pharmaceutically acceptable ester, stereoisomer, or tautomer thereof, may be an amount sufficient to decrease activity of anti-apoptotic Syk proteins. For instance, in some embodiments, a Syk inhibitor as disclosed herein is administered to a human at a dose resulting in about 50%, about 55%, about 60%, about 65%, about 70%, about 75%, about 80%, about 90%, about 95%, or about 99% Syk target inhibition.

In some embodiments, the therapeutically effective amount of a Syk inhibitor as disclosed herein is a dose corresponding to 1 nmol to 200 nmol of the Syk inhibitor used in an apoptosis assay run with 10% serum.

### EXAMPLE

The following study was conducted to evaluate the efficacy of filgotinib in combination with a Syk Inhibitor in a recognized model of arthritis, the rat type II collagen-induced arthritis model.

### Methods

Female Lewis rats (Charles River) were housed 3-4/cage in shoe-box polycarbonate cages with wire tops, wood chip bedding, and suspended food and water bottles. Animals were acclimated for 10 days prior to being immunized with type II collagen. An attending veterinarian was on site or on call during the live phase of the study. No concurrent medications were given. During the acclimation and study periods, animals were housed in a laboratory environment with temperatures ranging 67-76° F. and relative humidity of 30%-70%. Automatic timers provided 12 hours of light and 12 hours of dark. Animals were allowed access ad libitum to Harlan Teklad Rodent Chow and fresh municipal tap water.

Animals (n=8 rats/group for arthritis) were anesthetized with isoflurane and injected intradermally/subcutaneously (ID/SC) with 400 μl of Freund's Incomplete Adjuvant (Sigma Aldrich) containing 2 mg/ml porcine type II collagen (Chondrex) at 2 sites (200 μl per site) at the base of the tail on Day 0 and then 100 μl in one site at the base of the tail on Day 7. Animals were enrolled in treatment groups on Study Day 13 and were randomized such that each group had approximately equal mean ankle caliper measurements. Animals were weighed on Study Days 9 and 13-34. Dosing of the study compounds was initiated on Study Day 17 and continued for 17 days. Rats were euthanized for necropsy on Study Day 34.

Filgotinib, a Syk inhibitor (Formula ((Ib)), and a combination of filgotnib and Formula (Ib) were administered orally to rats in a vehicle containing Cremophor EL® (20%), ethanol (10%), and filtered water (70%). Filgotinib was administered at 3 mg/kg of animal body mass, and Formula (Ib) was administered at 10 mg/kg of animal body mass, both alone and in combination, based on the latest body weight measurements of the animals.

Caliper measurements of right and left ankle diameters were taken on Study Days 9 (Day −1 of arthritis) and 13-34. Ankle caliper measurements were made with a Digitrix II micrometer (Fowler & NSK). Baseline measurements were taken using one ankle with values rounded to one thousandth of an inch. Measurements were confirmed as clinically normal (0.260-0.264 in) by comparison with historical values for rats based on a range of body weights. Baseline measurements were then applied to both ankles, and these values remained with the animal so long as the ankle was clinically normal with good definition of all the ankle bones and no evidence of inflammation.

### Results

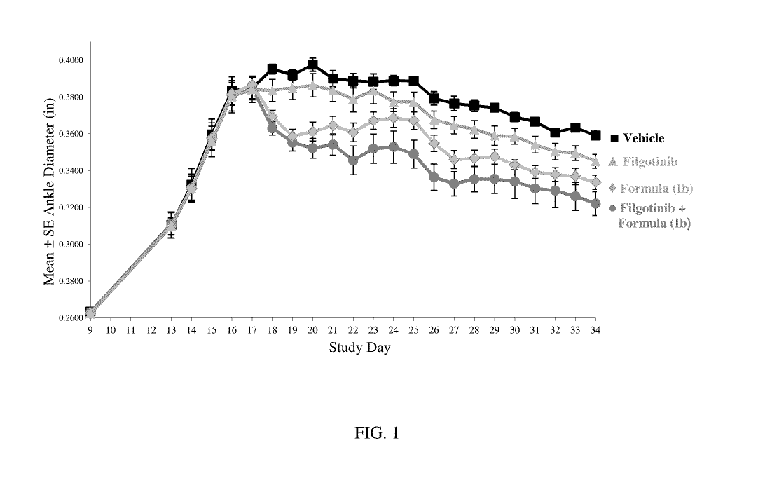

Mean daily ankle diameter measurements for test animals are shown in FIG. 1. The combination of filgotnib (3 mg/kg)+Formula (Ib) resulted in a significant reduction of ankle diameter toward normal on Study Days 18-34 relative to treatment with vehicle.

1. A method of treating an inflammatory disorder in a human in need thereof, comprising administering to the human:
(i) a therapeutically effective amount of filgotinib or a pharmaceutically acceptable salt, solvate, polymorph, or metabolite thereof; and
(ii) a therapeutically effective amount of a Syk inhibitor.

2. The method of claim 1, wherein the Syk inhibitor is entospletinib or a pharmaceutically acceptable salt, solvate, or polymorph thereof.

3. The method of claim 1, wherein the Syk inhibitor is a compound of Formula (I): 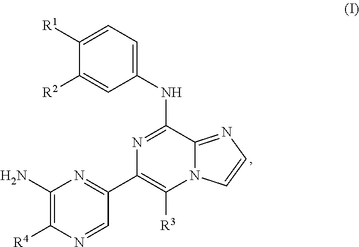
or a pharmaceutically acceptable salt, pharmaceutically acceptable co-crystal, pharmaceutically acceptable ester, stereoisomer, or tautomer thereof,
wherein:
R¹ is: 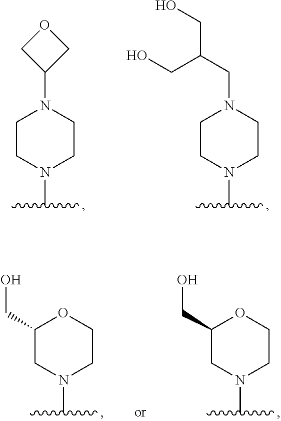 [SMILES: *N1CCN(C2COC2)CC1 ; *N1CCN(CC(CO)CO)CC1 ; *N1CCO[C@@H](CO)C1 ; *N1CCO[C@H](CO)C1]
wherein [symbol] indicates the point of attachment to the remainder of the compound of Formula (I),
R² is H or 2-hydroxyethoxy,
R³ is H or methyl, and
R⁴ is H or methyl.

4. The method of claim 3, wherein the Syk inhibitor is a compound of Formula (Ia): 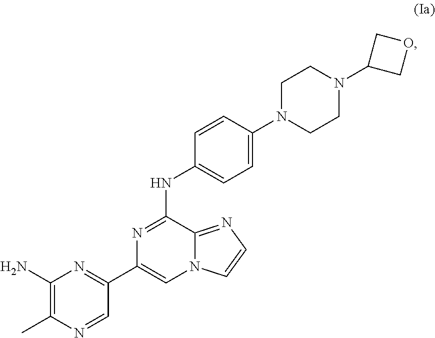 [SMILES: Cc1ncc(-c2cn3ccnc3c(Nc3ccc(N4CCN(C5COC5)CC4)cc3)n2)nc1N]
or a pharmaceutically acceptable salt, pharmaceutically acceptable co-crystal, pharmaceutically acceptable ester, stereoisomer, or tautomer thereof.

5. The method of claim 3, wherein the Syk inhibitor is a compound of Formula (Ib): 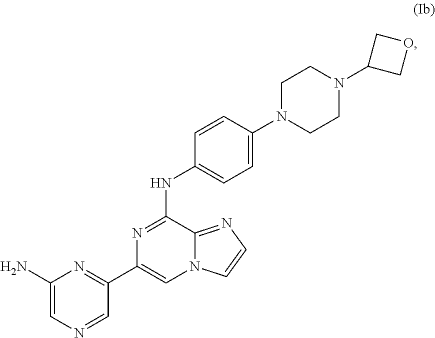 [SMILES: Nc1cncc(-c2cn3ccnc3c(Nc3ccc(N4CCN(C5COC5)CC4)cc3)n2)n1]
or a pharmaceutically acceptable salt, pharmaceutically acceptable co-crystal, pharmaceutically acceptable ester, stereoisomer, or tautomer thereof.

6. The method of claim 3, wherein the Syk inhibitor is a compound of Formula (Ic): 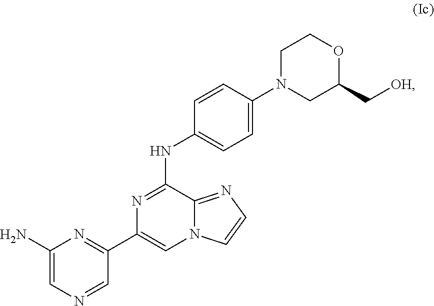 [SMILES: Nc1cncc(-c2cn3ccnc3c(Nc3ccc(N4CCO[C@@H](CO)C4)cc3)n2)n1]
or a pharmaceutically acceptable salt, pharmaceutically acceptable co-crystal, pharmaceutically acceptable ester, stereoisomer, or tautomer thereof.

7. The method of claim 3, wherein the Syk inhibitor is a compound of formula (Id): 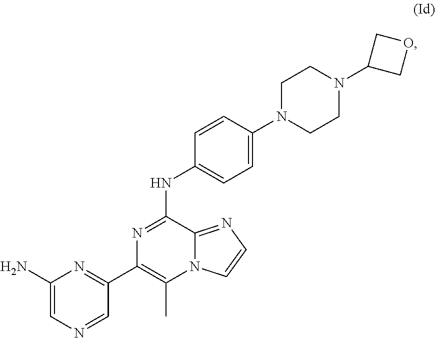 [SMILES: Cc1c(-c2cncc(N)n2)nc(Nc2ccc(N3CCN(C4COC4)CC3)cc2)c2nccn12]
or a pharmaceutically acceptable salt, pharmaceutically acceptable co-crystal, pharmaceutically acceptable ester, stereoisomer, or tautomer thereof.

8. The method of claim 3, wherein the Syk inhibitor is a compound of Formula (Ie): 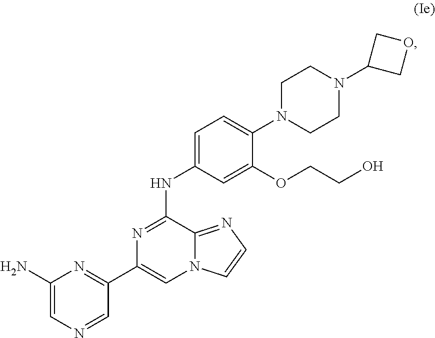 [SMILES: Nc1cncc(-c2cn3ccnc3c(Nc3ccc(N4CCN(C5COC5)CC4)c(OCCO)c3)n2)n1]
or a pharmaceutically acceptable salt, pharmaceutically acceptable co-crystal, pharmaceutically acceptable ester, stereoisomer, or tautomer thereof.

9. The method of claim 3, wherein the Syk inhibitor is a compound of Formula (If): 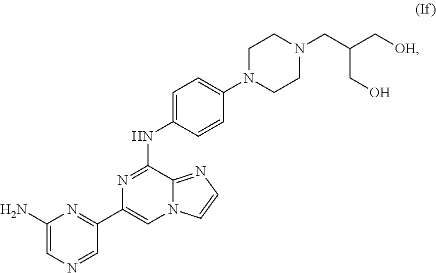 [SMILES: Nc1cncc(-c2cn3ccnc3c(Nc3ccc(N4CCN(CC(CO)CO)CC4)cc3)n2)n1]
or a pharmaceutically acceptable salt, pharmaceutically acceptable co-crystal, pharmaceutically acceptable ester, stereoisomer, or tautomer thereof.

10. The method of claim 3, wherein the Syk inhibitor is a compound of Formula (Ig): 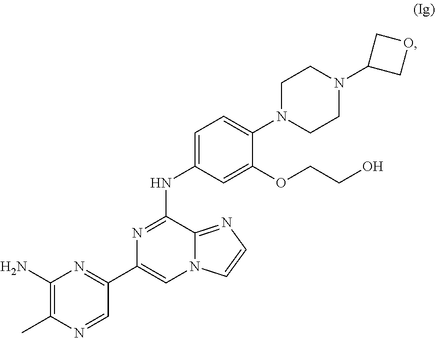 [SMILES: Cc1ncc(-c2cn3ccnc3c(Nc3ccc(N4CCN(C5COC5)CC4)c(OCCO)c3)n2)nc1N]
or a pharmaceutically acceptable salt, pharmaceutically acceptable co-crystal, pharmaceutically acceptable ester, stereoisomer, or tautomer thereof.

11. The method of claim 1, wherein the inflammatory disorder is systemic lupus erythematosus, graft versus host disease, myestenia gravis, rheumatoid arthritis, acute disseminated encephalomyelitis, idiopathic thrombocytopenic purpura, multiple sclerosis, Sjogren's syndrome, psoriasis, autoimmune hemolytic anemia, asthma, ulcerative colitis, Crohn's disease, irritable bowel disease, or chronic obstructive pulmonary disease.

12. The method of claim 1, wherein the inflammatory disorder is rheumatoid arthritis and the Syk inhibitor is a compound of Formula (Ib): 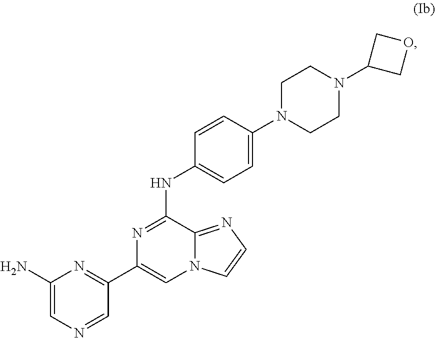 [SMILES: Nc1cncc(-c2cn3ccnc3c(Nc3ccc(N4CCN(C5COC5)CC4)cc3)n2)n1]
or a pharmaceutically acceptable salt, pharmaceutically acceptable co-crystal, pharmaceutically acceptable ester, stereoisomer, or tautomer thereof.

13. The method of claim 1, wherein the inflammatory disorder is systemic lupus erythematosus and the Syk inhibitor is a compound of Formula (Ib): 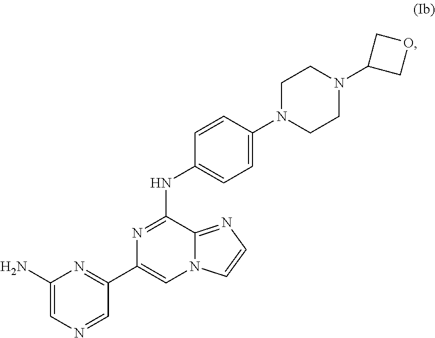 [SMILES: Nc1cncc(-c2cn3ccnc3c(Nc3ccc(N4CCN(C5COC5)CC4)cc3)n2)n1]
or a pharmaceutically acceptable salt, pharmaceutically acceptable co-crystal, pharmaceutically acceptable ester, stereoisomer, or tautomer thereof.

14. The method of claim 1, wherein the inflammatory disorder is graft versus host disease and the Syk inhibitor is entospletinib or a pharmaceutically acceptable salt, solvate, or polymorph thereof.

15-19. (canceled)

20. A co-formulation, comprising:
(i) filgotinib or a pharmaceutically acceptable salt, solvate, polymorph, or metabolite thereof;
(ii) a Syk inhibitor; and
(iii) a pharmaceutically acceptable carrier.

21. The co-formulation of claim 20, wherein the Syk inhibitor is entospletinib pharmaceutically acceptable salt, solvate, or polymorph thereof.

22. The co-formulation of claim 20, wherein the Syk inhibitor is a compound of Formula (I): 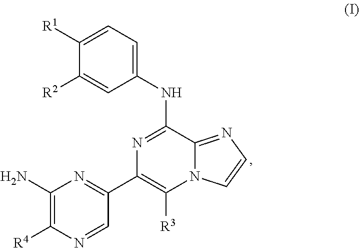
or a pharmaceutically acceptable salt, pharmaceutically acceptable co-crystal, pharmaceutically acceptable ester, stereoisomer, or tautomer thereof,
wherein:
R¹ is: 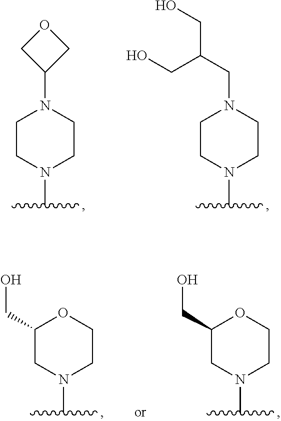 [SMILES: *N1CCN(C2COC2)CC1 ; *N1CCN(CC(CO)CO)CC1 ; *N1CCO[C@@H](CO)C1 ; *N1CCO[C@H](CO)C1]
wherein [symbol] indicates the point of attachment to the remainder of the compound of Formula (I),
R² is H or 2-hydroxyethoxy,
R³ is H or methyl, and
R⁴ is H or methyl.

23. The co-formulation of claim 20, wherein the Syk inhibitor is: 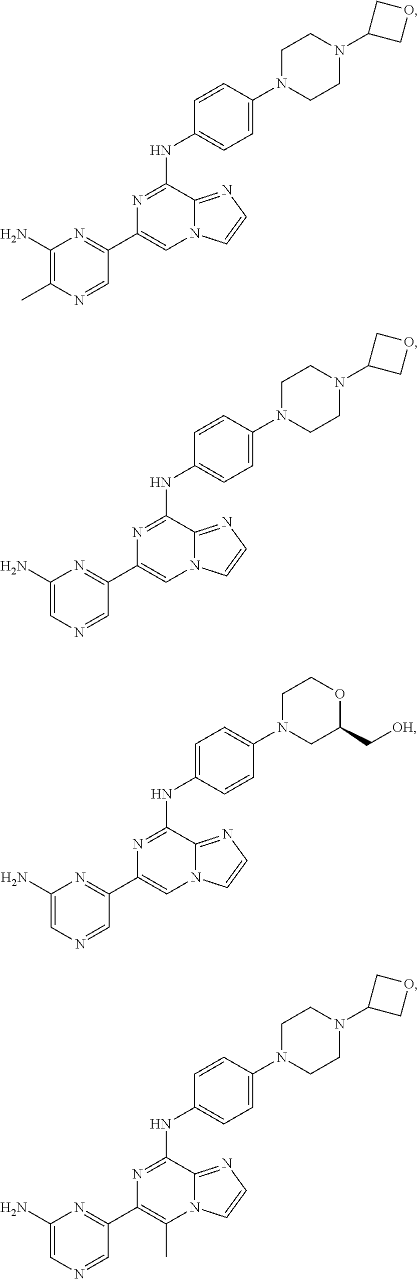 [SMILES: Cc1ncc(-c2cn3ccnc3c(Nc3ccc(N4CCN(C5COC5)CC4)cc3)n2)nc1N ; Nc1cncc(-c2cn3ccnc3c(Nc3ccc(N4CCN(C5COC5)CC4)cc3)n2)n1 ; Nc1cncc(-c2cn3ccnc3c(Nc3ccc(N4CCO[C@@H](CO)C4)cc3)n2)n1 ; Cc1c(-c2cncc(N)n2)nc(Nc2ccc(N3CCN(C4COC4)CC3)cc2)c2nccn12] 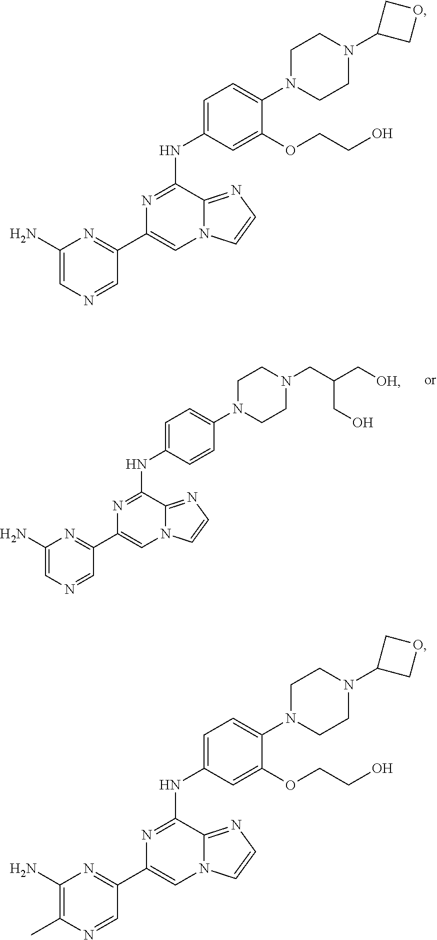 [SMILES: Nc1cncc(-c2cn3ccnc3c(Nc3ccc(N4CCN(C5COC5)CC4)c(OCCO)c3)n2)n1 ; Nc1cncc(-c2cn3ccnc3c(Nc3ccc(N4CCN(CC(CO)CO)CC4)cc3)n2)n1 ; "or" ; Cc1ncc(-c2cn3ccnc3c(Nc3ccc(N4CCN(C5COC5)CC4)c(OCCO)c3)n2)nc1N]
or a pharmaceutically acceptable salt, pharmaceutically acceptable co-crystal, pharmaceutically acceptable ester, stereoisomer, or tautomer thereof.

24. The co-formulation of claim 20, wherein the Syk inhibitor is a compound of Formula (Ib): 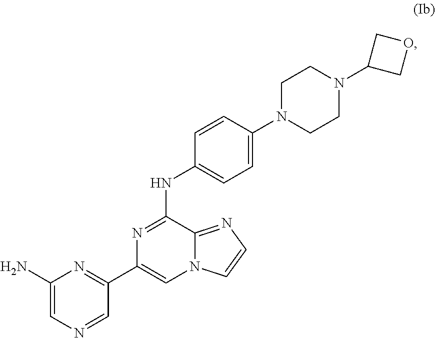 [SMILES: Nc1cncc(-c2cn3ccnc3c(Nc3ccc(N4CCN(C5COC5)CC4)cc3)n2)n1]
or a pharmaceutically acceptable salt, pharmaceutically acceptable co-crystal, pharmaceutically acceptable ester, stereoisomer, or tautomer thereof.

25-29. (canceled)
